(19) United States
(12) Patent Application Publication
(10) Pub. No.: US 2022/0298100 A1
(43) Pub. Date: Sep. 22, 2022

(54) DISTILLED CASHEW NUT SHELL LIQUID BASED, WATER THINABLE PHENALKAMINE AS CURING AGENT FOR EPOXY PAINT COMPOSITIONS

(71) Applicants: JAYRAM MANGESH NADKARNI, MUMBAI (IN); PRASAD JAYRAM NADKARNI, MUMBAI (IN)

(72) Inventors: JAYRAM MANGESH NADKARNI, MUMBAI (IN); PRASAD JAYRAM NADKARNI, MUMBAI (IN)

(21) Appl. No.: 15/733,683

(22) PCT Filed: Sep. 27, 2020
(86) PCT No.: PCT/IB2020/059029

§ 371 Date: Sep. 29, 2020

(30) Foreign Application Priority Data
Dec. 13, 2019 (IN) 201921051663

(51) Int. Cl. C07C 209/60; C08G 59/64; C09D 163/00

(52) U.S. Cl. CPC C07C 209/60; C08G 59/64; C09D 163/00

(57) ABSTRACT

A water-Soluble phenalkamine curing agent including a water-soluble polyamine which is a reaction product of: (a) an unsaturated dicarboxylic acid modified distilled CNSL molecule; and (b) a water-soluble polyamine with aldehyde. The water soluble Mannich base is a reaction product of a polyamine containing at least two amino groups with a phenolic compound, with aid of an aldehyde. The dicarboxylic acid modified distilled CNSL intermediate is produce by diel alder reaction on side chain of cardanol which is renewable biomass.

### FIELD OF INVENTION

This invention relates to water-Soluble phenalkamine curing agent, paint compositions thereof with epoxy resins and methods for making the same, and more particularly relates to water thinable phenalkamine as curing agent in aqueous solution for curing of epoxy resin-based compositions.

### PRIOR WORKS OF ART

Most organic Solvents are ecologically unfriendly and damaging for the environment. Attempts were made to replace the organic Solvents by water. Water soluble phenalkamine produced was tested for corrosion resistance and other properties after curing with epoxy resin which shows excellent performance. No prior works of the prior art that closely or partly resemble the work disclosed in the present invention are present.

### OBJECTIVES OF THE PRESENT INVENTION

One objective of the present invention is to provide an unsaturated dicarboxylic acid modified distilled CNSL by Diels alder reaction on side chain unsaturation. This phenolic compound intermediate is used to produce water soluble phenalkamine.

Another objective of the present invention is to provide a water soluble phenalkamine via Mannich reaction of phenolic intermediate with polyamine and aldehyde. Phenalkamine produce was tested for corrosion resistance and performance when cured with epoxy resin.

### SUMMARY OF THE INVENTION

This invention related to two components water-based epoxy coatings based upon liquid epoxy resin cured with unsaturated dicarboxylic acid modified distilled CNSL based phenalkamine. Regulatory requirements minimizing the use of Volatile Organic Compound (VOC) containing coatings are becoming more stringent. One approach to minimizing the level of VOC for epoxy coatings has been use of water based epoxy systems. These products can provide desirable performance including fast dry time, good corrosion resistance, good pot life, low odour and can replace traditional solvent based epoxy polyamide/polyamine/phenalkamine coatings in high performance industrial applications.

The water soluble phenalkamine comprises reaction product of A) unsaturated dicarboxylic acid modified distilled CNSL via Diels alder and B) polyamine and aldehyde with active hydrogen to from Mannich base. Resultant phenalkamine provides stable water-thinable epoxy curing agent composition in aqueous media. The water-thinable composition produce in this invention are readily soluble in aqueous media without use of any surfactant or external additive.

The resultant phenalkamine composition is readily soluble in water and capable of dispersing liquid epoxy resin in aqueous media. Thus, another embodiment of this invention is curable coating composition comprising water soluble phenalkamine curing agent and liquid epoxy resin.

### DESCRIPTION OF DRAWINGS

FIG. 1 illustrates a composition of distilled cashew nut shell liquid; and

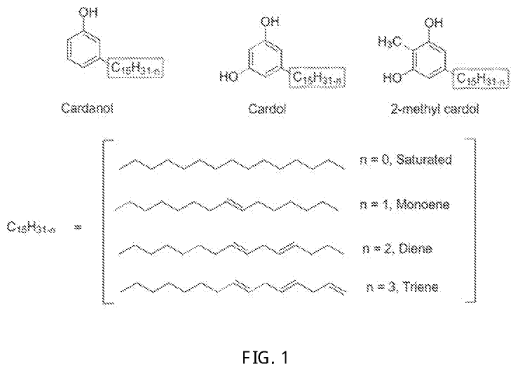

FIG. 2 illustrates Diels alder reaction of unsaturated dicarboxylic acid with Distilled CNSL side chain unsaturation.

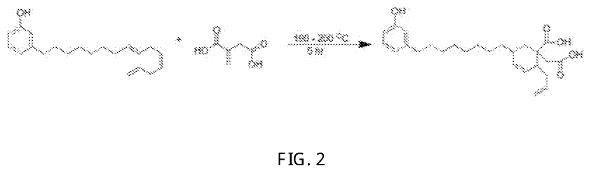

### DETAILED DESCRIPTION OF THE PREFERRED EMBODIMENTS

The invention herein disclosed was driven by the desire to develop a stable water thinable phenalkamine composition for curing epoxy resins. The invention flows from the surprising discovery that a water-thinable composition comprising a water thinable phenalkamine forms a solution with water and not an emulsion or dispersion, provided that (1) the water-thinable phenalkamine is formed by reacting: (a) a dicarboxylic acid modified distilled CNSL intermediate by Diels alder reaction with side chain at unsaturation and (b) a Mannich reaction with polyamine with aldehyde.

It is discovered that, the water-thinable phenalkamine of the present invention can be diluted with water to at least 50 wt. % and preferably to at least 10 wt. % to form a single phase at a temperature of 25° C., provided that water is essentially free of an organic co-Solvent and minerals. The water-based Solutions of phenalkamine of the present invention are capable of maintaining a single-phase uniformity required for further application or a storage of the Solution.

Preferably, the water-thinable composition of the present invention and the paint composition of the present invention have a 0% VOC, which enhances their application in a closed environment wherein using Solvents can present a fire hazard or be harmful to a user. Also due to restriction on use of volatile solvent users are shifting to solvent less and waterborne systems. Phenalkamine curing agents are basically applied for high degree of corrosion resistance, this invention provides water soluble phenalkamine composition when used for curing epoxy resin provides good drying performance and corrosion resistance.

The cashew nut shell liquid is obtained as a by-product of cashew nut processing extracted from shell of cashew nut Distilled CNSL is natural and renewable biomass phenolic material. The cardanol is distilled product of CNSL with content at least 80 weight % to 100 weight % of the cashew nutshell liquid, based on a total weight of component. The distilled product of CNSL mainly includes cardanol as a primary component and additionally include cardol, methylcardol, as secondary components. The composition of cardanol varies based on degree of unsaturation inside chain. Cardanol is mixture of tri-unsaturated cardanol (41%) which is major component, 34% mono-unsaturated, 22% bi-unsaturated, and 2% saturated.

The water-thinable phenalkamine curing agent is synthesised in two step, a) preparation of intermediate of distilled CNSL and dicarboxylic acid and b) Mannich reaction of intermediate phenolic compound with polyamine with aid of aldehyde to produce final product

The unsaturation present on the side chain of cardanol may be utilized for purposes of grafting unsaturated compound. for example, cardanol may be grafted onto an unsaturated reactant such as an unsaturated polyol, an unsaturated polycarboxylic acid or anhydride, or any other unsaturated compound having suitable reactivity to form a grafted cardanol intermediate. Any suitable grafting mechanism may be used to produce the grafted adduct or graft polymer, including mechanisms such as Diels-Alder or Diels-ene addition reactions. A presently preferred distilled CNSL intermediate is a reaction product of distilled CNSL and an unsaturated dicarboxylic acid. Suitable unsaturated dicarboxylic acid may include unsaturated dicarboxylic acid anhydrides such as maleic anhydride, itaconic anhydride, nonenylsuccinic anhydride, citraconic anhydride, itaconic acid, maleic acid, nonenylsuccinic acid and mixtures thereof. The distilled CNSL intermediate is produce by reaction of unsaturated dicarboxylic acid with unsaturation on side chain of cardanol. The conditions of the reaction are controlled, including the temperature, to avoid gelling. The resulting carboxylic functional intermediate is reacted with polyamine and formaldehyde to produce phenalkamine via Mannich reaction. The Mannich base is prepared by reaction of phenolic compound, an aldehyde and polyamine. Reaction takes place by elimination of water molecule producing linkage between phenolic compound and polyamine via methylene linkage. The carboxylic function available on backbone are neutralized by amine to produce an aqueous solution.

In preparation of phenalkamine curing agent formaldehyde is used for condensation. Other aldehyde which can be used are acetaldehyde, furfuraldehyde and alike.

The polyamine used for producing water soluble phenalkamine are selected from aliphatic, aromatic or alicyclic polyamine and mixture thereof. The Polyamine is preferably ethylene diamine (EDA), diethylene triamine (DETA), triethylenetetramine (TETA), Xylenediamine, 1,3-bis(aminomethyl)cyclohexane, and mixtures thereof can be used for synthesis. Polyamine containing oxypropylene unit are used to improve water solubility of phenalkamine. Preferably polyether amine with molecular weight 230, 430, 2000 and 4000 can be used in combination with other polyamines. The amino alkyl group is preferably an amino methyl, amino ethyl, amino propyl or amino butyl, wherein the alkyl group is either a straight chain or branched. More preferably, the amino alkyl group is amino methyl or amino ethyl.

The resulting carboxylic-functional on distilled CNSL intermediate is neutralized using a base such as a tertiary amine to produce a water soluble phenalkamine. Some of the commonly used amines are triethylamine, N,N dimethyl benzyl amine, triethanol amine, 2,4,6-tris(dimethyaminomethyl)phenol, ammonia and its derivative and alike.

### TEST METHODS

Unless indicated otherwise, the following test methods were utilized in the Examples that follow.

### 1. Acid Value

Acid value is evaluated according to ASTM D 1980 titration method, indicated as mg KOH/gm.

### 2. Viscosity

Viscosity is measured according to ASTM D 2196 with Brookfield digital viscometer LVDV model having multiple rotational speed. When not mentioned parameters are spindle number 63, rotation speed 30 RPM and temperature 25° C. for testing.

### 3. Solid Content

Solid content mentioned was tested following ASTM D1259. Volatile matter present in sample was evaporated by heating in oven for one hour and solid content is calculated as percentage comparing weight of sample before and after heating.

### 4. Drying Performance

Phenalkamine produced are cross linked with liquid epoxy resin EEW 190 to test performance properties of cured film. ASTM D 5895 was followed to record test results. Drying performance are tested at 25° C. and 5° C. with 40 to 45|m DFT on B. K. Drying recorder.

### 5. Adhesion Test

Adhesion testing was performed to assess whether the coating compositions adhere to the coated substrate. The Adhesion Test was performed according to ASTM D 3359-Test method.
The results are rated as follows:
A: No abnormalities,
B: Peelings on part of the top coating surface,
C: Peelings on the entire top coating surface

### 6. Corrosion Resistance

The corrosion resistance was check with standard method ASTM B117. The test panel were prepared and subjected to salt spray conditions and evaluated for rusting and blisters. The condition of the coating Surface was rated as follows:
A: No abnormalities,
B: Partially rusted or blistered,
C: Entirely rusted or blistered
The results of this test for coatings prepared according to the present invention are presented in Tables 3.

### Example 1: Synthesis of Unsaturated Dicarboxylic Acid Modified Distilled CNSL Intermediate

Distilled CNSL side chain was modified with unsaturated dicarboxylic acid via Diels alder reaction. In particular, 70 grams of distilled CNSL, 30 grams of itaconic acid are charged in four necked round bottom flask equipped with a condenser, thermometer, a mechanical stirrer, and a nitrogen connection to form a reaction mixture. Raise the temperature of reaction mixture 190-200° C. and maintain for 5 hr to produce intermediate. Tested for acid value 73 mg KOH/gm, viscosity @25° C. 980 cps.

### Example 2: Synthesis of Water Soluble Phenalkamine Curing Agent

The phenolic intermediate synthesis in example 1 was reacted with polyamine and formaldehyde to undergo Mannich reaction to produce water soluble phenalkamine. In particular, 50 grams of intermediate from example 1, 25-gram polyether amine with molecular weight 230, and 14 grams of DETA are charged in four-necked round bottom flask equipped with a condenser, thermometer, a mechanical stirrer, and a nitrogen connection and agitated to form homogeneous mixture. Raise the temperature of reaction mixture 60-70° C. add 5-gram paraformaldehyde under agitation. after complete addition of paraformaldehyde maintain temperature of reaction mass between 70 to 80° C. for 1 hr.

Add 2,4,6-tris(dimethyaminomethyl)phenol 5 gram to reaction mixture. Maintain reaction mixture for 4-5 hr under agitation. Raise the temperature of reaction mixture to 130 to 140° C. and remove water of condensation. Phenalkamine synthesised was tested for amine value 280 mg/KOH, viscosity @25° C.10,000 cps.

The paint compositions prepared are tested for drying, corrosion resistance, adhesion, storage stability, and over coating performance (see table 3).

A metal plate measuring 0.8×100×150 mm is Sand by 400 emery paper and after that spray coated with the water-thinable composition to a dry film thickness of about 40 to 45|m, and the coating is dried at 25° C. and 65% RH for 7 days. The coating is further spray coated with each of the top coating compositions shown in Table-2 to a dry film thickness of about 40 to 45|m, and the coating is dried at 25° C. and 65% R H giving a test specimen.

### Example 3: Paint Composition Based on Waterborne Phenalkamine

All of the Base ingredients were combined in the high-speed dissolver. The ingredients of Part A were combined according to the order reflected in Table 1. Once all of the ingredients were combined, the ingredients were Mix in a high-speed dissolver until a smooth finish on panel was achieved. Once this smooth finish was achieved, the temperature of the blend was brought to approximately 120 ^{é}F (approximately 48.9° C.) and held for approximately 20 minutes while the ingredients were continuously agitated.

Part B was then prepared by combining the ingredients reflected in Table 1. under agitation. Then, Part B was kept separate.

At the time of coating application Mix. the Base & Hardener part as per weight ratio Base Part-64.61 gms. and Hardener Part 35.39 gms.

TABLE 1
Coating Paint Compositions.

| Description | Example-3 |
|---|---|
| Part ⁻A (Epoxy Base) | |
| ¹Epoxy Resin (Ep-Eq.-190) | 25.20 |
| ²Pepox-7513 | 10.81 |
| ³Nuosper-657 | 0.30 |
| ⁴Titanium Di-oxide | 6.00 |
| ⁵Carbon black Haf-330 | 0.015 |
| ⁶Barytes (20 micron) | 16.00 |
| ⁷Silica (20 micron) | 5.285 |
| ⁸Namlon T-206 | 0.600 |
| ⁹Mpax-60 | 0.400 |
| Part-B (waterborne Hardener) | |
| Example 2 Resin | 17.63 |
| ¹⁰Pcat-1030 | 2.00 |
| ¹¹Disperlon AQ-160 | 1.00 |
| Water | 14.76 |

### Example 4 & 5: Paint Composition of Epoxy and Polyurethane (Two Pack) for Over Coating

All of the ingredients were combined in the Mill. In Examples, the ingredients of Part A were combined according to the order reflected in Table 2. Once all of the ingredients were combined, the ingredients were grind at Mill until a Hegman reading greater than 7.00 was achieved. Once this Hegman reading was achieved, the temperature of the blend was brought to approximately 120^{é}F (approximately 48.9° C.) and held for approximately 20 minutes while the ingredients were continuously agitated. Next, Part B was prepared by combining the ingredients under agitation in the order reflected in Table. Part B is added slowly to grinding machine for flushing after flushing add the flush material into Part A under agitation. Part C is prepared by combining the ingredients under agitation in the order reflected in Table 2. Part C is added slowly to Part A under agitation.

Part D is then prepared by combining the ingredients reflected in Table 2 under agitation. Then, Part D was added under agitation at the time of application to the already combined blend of Parts A, B. & C.

Coating Compositions was prepared for over coating using the components and amounts identified i n Table 2.

| Description | Example 4 | Example 5 |
|---|---|---|
| Part ⁻A | | |
| ¹² Epoxy Resin (Ep-Eq.-480) | 12.00 | — |
| ¹³Acrylic Polyol (OH-Value 50) | — | 12.00 |
| ¹⁴Urea Formaldehyde Resin | 1.00 | — |
| Nuosper-657 | 0.30 | 0.30 |
| Ethyl Cello solve Acetate | — | 1.5 |
| Xylene | 8.00 | 8.00 |
| Butanol | 1.5 | — |
| MIBK | 1.5 | — |
| Titanium Di-oxide | 16.00 | 19.00 |
| ¹⁵Bent one Jelly 10% in Xylene. | 7.00 | 2.00 |
| Part-B | | |
| Xylene | 4.00 | 1.5 |
| Butanol | 1.50 | — |
| Ethyl Cello solve Acetate | — | 1.5 |
| Part-C | | |
| Epoxy Resin (Ep-Eq.-480) | 33.84 | — |
| Xylene | 4.02 | — |
| MIBK | 1.50 | — |
| ¹³Acrylic Polyol (OH-Value 50) | — | 43.00 |
| Ethyl Cello solve A cetate | — | 1.20 |
| Part-D | | |
| ¹⁵Polyamide-125 | 7.84 | — |
| ¹⁶Aliphatic Isocyanate | — | 7.84 |
| Butyl Acetate | — | 2.16 |

TABLE 3
Physical & Chemical properties paint composition

| Physical Properties | Example-3 | |
|---|---|---|
| Viscosity on Brookfield. @30éC | 5150 | cps |
| B.K. Drying Time @ 25éC | | |
| Touch Dry | 5 | Hrs |
| Hard Dry | 9 | Hrs. |
| B.K. Drying Time @ 5éC | | |
| Touch Dry | 18 | Hrs |
| Hard Dry | 40 | Hrs. |
| Solid content at 120éC/1 hr | 83.00% | |
| Density .as per ASTM D-1475-98 | 1.213 | |
| Performance properties | | |
| Salt Spray Resistance as per ASTM-B-117 at 45 \| m | A | |
| DFT on Mild Steel surface, after 400 Hrs. | | |
| Adhesion On Mild steel surface. | A | |
| Over Coating Adhesion @ 50 \| m DFT | | |
| With I).2K Epoxy systems @ 40 \| m | A | |
| With II).2K PU systems @ 40 \| m | A | |
| Pot Life @30 éC (100 gm.) | 30 | Min. |

### BEST METHOD OF OPERATING THE INVENTION:

1. Best method for the manufacture of distilled CNSL modified with itaconic acid is described in examples 1.
2. Best method for the manufacture of water soluble phenalkamine is described in examples 2.
3. Best method for preparation of water soluble phenalkamine paint composition is described i n example 3.

We claim:

1. A process for the production of water thinable phenalkamine curing agent for epoxy resins, comprising:
a. modifying distilled cashew nut shell liquid with unsaturated dicarboxylic acid at the side chain unsaturation of cardanol by Diels alder reaction to obtain phenolic intermediate;
b. converting the said phenolic intermediate in step a) into water-thinable phenalkamine curing agent for epoxy resins by Mannich reaction of reacting the said phenolic intermediate with polyamine and paraformaldehyde to obtain water-thinable phenalkamine curing agent; and
c. diluting the said water-thinable phenalkamine curing agent of step (b) in demineralized and solvent-freed water to dilution between 10% to 50% by weight, wherein said water thinable phenalkaline curing agent for epoxy resins is in stable solution form.

2. The process as claimed in claim 1, wherein the said distilled cashew-nut-shell-liquid is a mixture of tri-unsaturated cardanol in 41 weight % as a major component, mono-unsaturated cardanol in 34 weight %, bi-unsaturated cardanol in 22 weight % and saturated cardanol in 2 weight %, based on the total weight of the said distilled cashew-nut-shell liquid.

3. The process as claimed in claim 1, wherein the said Diel alder reaction is carried out, keeping ratio of distilled cashew nut shell liquid to itaconic acid at 70:30 by weight and the reaction temperature between 190° C. to 200° C.

4. The process as claimed in claim 1, wherein the said phenolic compound intermediate is further reacted with polyamine and paraformaldehyde in Mannich reaction at reaction temperature between 60° C. and 130° C. until the said phenalkamine is water thinable.

5. The process as claimed in claim 1, wherein suitable unsaturated dicarboxylic acid for modifying distilled cashew nut shell liquid at the side chain unsaturation of cardanol by Diels alder reaction may include unsaturated dicarboxylic acid, wherein said unsaturated dicarboxylic acid includes unsaturated dicarboxylic acid anhydrides selected from maleic anhydride, itaconic anhydride, nonenylsuccinic anhydride, citraconic anhydride, maleic acid, nonenylsuccinic acid and mixtures thereof.

6. The process as claimed in claim 1, wherein the said phenolic compound intermediate may be reacted by Mannich reaction for condensation with formaldehyde, acetaldehyde and furfuraldehyde.

7. The process as claimed in claim 1, wherein the said polyamine for Mannich reaction may include aliphatic, aromatic, alicyclic polyamines and mixtures thereof, including ethylene diamine, diethylene triamine, triethylene tetramine, xylene diamine, 1,3-bis(aminomethyl)cyclohexane, and mixtures thereof.

8. The process as claimed in claim 7, wherein the said polyamines containing oxypropylene unit are used to improve water solubility of phenalkamine.

9. The process as claimed in claim 8, wherein the said polyamines having molecular weight of 230, 430, 2000 and 4000 are used in combination with the said other polyamines and the aminoalkyl group of the said polyamines is amino methyl, amino ethyl, amino propyl or amino butyl in decreasing order of preference.

10. A method for production of water thinable phenalkamine curing agent for epoxy resins comprising:
a. charging 70 grams of distilled cashew nut shell liquid and 30 grams of itaconic acid into a four necked round bottom flask equipped with a condenser, thermometer, a mechanical stirrer and a nitrogen connection to form a reaction mixture;
b. raising the temperature of the said reaction mixture of step (a) to 190-200° C. and maintain the said temperature for five hours to produce intermediate until the said intermediate tests for acid value of 73 mg KOH/gm and viscosity @25° C. of 980 cps;
c. charging 50 grams of the said tested phenolic compound intermediate of step (b) with 25 grams of the said polyether amine having molecular weight of 230 and 14 grams of diethylene triamine into a four-necked round bottom flask equipped with a condenser, a thermometer, a mechanical stirrer and a nitrogen connection and agitating the contents to form a homogeneous mixture;
d. raising the temperature of the said reaction mixture of step (c) to 60-70° C.;
e. adding 5 grams of paraformaldehyde under agitation to the said heated mixture of step (d);
f. raising and maintaining, after complete addition of paraformaldehyde, the temperature of the said reaction mass between 70 to 80° C. for one hour;
g. adding 2,4,6-tris(dimethyaminomethyl)phenol weighing five grams to the said reaction mixture of step (f) and maintaining the said reaction mixture for 4-5 hr under agitation; and
h. raising the temperature of the said reaction mixture of step (g) to 130 to 140° C. and removing water of condensation until phenalkamine synthesised tests for amine value 280 mg/KOH, viscosity @ 25° C. 10,000 cps, wherein said water thinable phenalkamine curing agent for epoxy resins is in form of stable solution.
